(19) United States
(12) Patent Application Publication
(10) Pub. No.: US 2025/0332649 A1
(43) Pub. Date: Oct. 30, 2025

(54) MODIFIED BOW SAW

(71) Applicant: Gary Cantue, Beaumont, TX (US)

(72) Inventor: Gary Cantue, Beaumont, TX (US)

(21) Appl. No.: 18/646,360

(22) Filed: Apr. 25, 2024

(51) Int. Cl. B23D 51/10; A61B 17/14; B23D 49/16; B23D 51/14

(52) U.S. Cl. CPC B23D 51/10; B23D 49/162; B23D 51/14; A61B 17/142

(57) ABSTRACT

A modified bow saw includes a frame including two ends, a spring holder attached to each end of the frame; the spring holders each including a spring mechanism, a saw blade including two ends, each end of the blade attached to the spring mechanisms, an adjustable depth bar guide attached to the frame, and an adapter configured to attach a spring to a circulating saw. The modified bow saw may include one or more ergonomic handles. The modified bow saw may include one or more braces connecting the frame with the spring holder.

### BACKGROUND

### Technical Field of the Invention

The invention is directed to the technical field of Bow Saws.

### Description of the Related Art

The bow saw is a versatile tool widely appreciated for its ability to cut through wood, bone, and various materials with ease. Its distinctive design, featuring a narrow, flexible blade held taut within a metal frame, allows for precise, controlled cuts. The bow saw excels at tasks like pruning, woodworking, and emergency situations in outdoor settings. However, the efficiency of the bow saw is compromised by its hand-operated nature. The manual operation of a bow saw can be physically demanding, especially during extended use or when cutting through denser materials. This places a considerable burden on the user in terms of both physical effort and the time required for completion. In professional or industrial contexts where speed and precision are paramount, powered alternatives such as chainsaws or reciprocating saws are favored for their capacity to streamline operations, but often result in uneven and rough cuts.

Accordingly, there exists a need for a means by which finesse and control of a bow saw can be made available in a powered version. The development of a modified, motorized, bowsaw fulfills this need.

### SUMMARY DISCLOSURE OF THE INVENTION

A modified bow saw includes a frame including two ends, a spring holder attached to each end of the frame; the spring holders each including a spring mechanism, a saw blade including two ends, each end of the blade attached to the spring mechanisms, an adjustable depth bar guide attached to the frame, and an adapter configured to attach a spring to a circulating saw. The modified bow saw may include one or more ergonomic handles. The modified bow saw may include one or more braces connecting the frame with the spring holder.

Other features and aspects will be apparent from the following detailed description, the drawings, and the claims.

### BRIEF DESCRIPTION OF THE DRAWINGS

FIG. 1 shows an embodiment of a bow saw of the disclosure.

FIG. 2 shows an adapter plate of the disclosure.

FIG. 3 shows another embodiment of a bow saw of the disclosure.

FIG. 4 shows an enlarged view of a rod holding spring of the disclosure.

FIG. 5 shows an example reciprocating saw of the disclosure.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DISCLOSURE OF THE INVENTION

The following detailed description is provided to assist the reader in gaining a comprehensive understanding of the methods, products, and/or systems, described herein. However, various changes, modifications, and equivalents of the methods, products, and/or systems described herein will be apparent to an ordinary skilled artisan.

### MODES FOR CARRYING OUT THE INVENTION

A modified or motorized bow saw or hacksaw can be constructed of a bow shaped frame, a saw blade, spring mechanisms, an adapter plate, an optional depth bar, optional braces, and optional ergonomic handles. The materials necessary to make the components are known to those skilled in the art and are readily available.

The modified Bowsaw is a bow saw that attaches to a reciprocating saw to provide power activation and back and forth sawing motion. It is configured as a conventional bow saw with a blade supported within, for example, a typical bow saw “C”-shaped bow. The present invention, however, provides several enhancements.

First, the blade is supported via a spring mechanism at each end where it connects to the bow. This allows the blade to move back and forth in a sawing motion while the bow remains stationary. Second, at least one spring mechanism can be provided with an adapter. The adapter attaches to both corded and battery-operated reciprocating saws, for example, using screw holes for attachment to the adapter. In this embodiment, the adapter may be a plate. This allows the reciprocating saw to drive the blade back and forth with the only requirement of the user to guide it. In addition, the invention may be equipped with a versatile adjustable depth bar guide, for use with varying material depths, ensuring accuracy and adaptability. Finally, the invention may be provided braces for vertical cuts and ergonomic handles for user comfort over long periods of use. These features allow the invention to handle materials of all sizes especially wood, quickens the cutting process, and allows for precision cuts. The use of the modified bow saw provides the control and precision of a bow saw, but with a motorized feature that speeds use and reduces user fatigue. This makes it an invaluable asset for woodworkers, construction professionals, and DIY enthusiasts alike.

The materials required to produce a motorized bow saw are readily available and well known to manufacturers of products of this type. The bow frame, adapter plate, depth bar, braces, and the like, could be made of steel. In embodiments, a stamping and forming operation could be used to make them. Such a process could utilize the design and use of custom dies. For larger scale production, suitable jigs and other assembly aids could be used to speed production and ensure consistency. After manufacture, a suitable protective finish such as paint or plating could be applied to prevent corrosion. The remaining components of the present invention such as the springs, fasteners, ergonomic handles, and the like, are generally commercially available. The relatively simple design of the invention and the materials of construction make the modified bow saw a cost-effective design due to the relatively low material and labor costs involved.

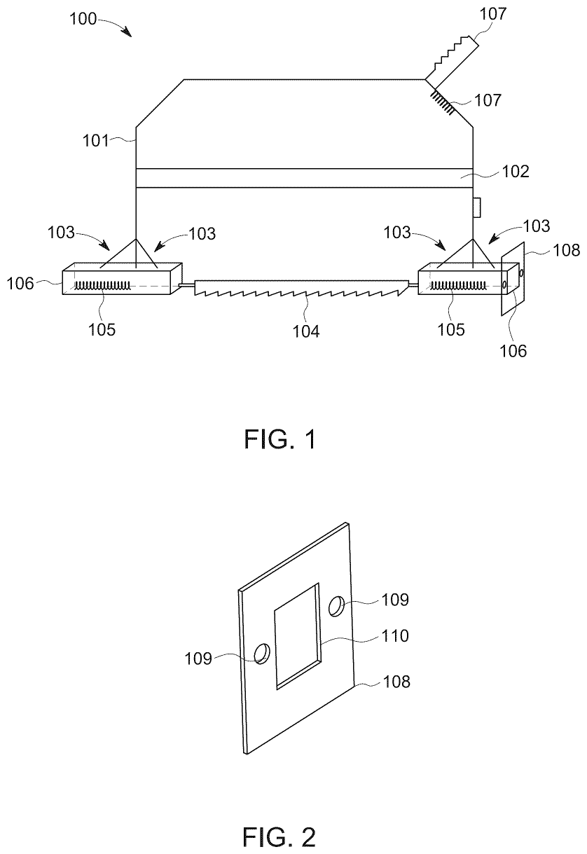

FIG. 1 shows a modified bow saw 100 of the disclosure. The modified bow saw includes frame 101, an adjustable depth bar guide 102 (which can be moved up and down); braces 103; cutting blade 104; Springs 105; spring holders 106 and adapter 108 (side view) for connecting to a reciprocating saw 8. FIG. 1 further shows optional ergonomic handles 107 attached to frame 2.

FIG. 2 shows a front view of adapter 108 including adapter holes 109 and opening 110. In embodiments, the adapter holes may be screw holes.

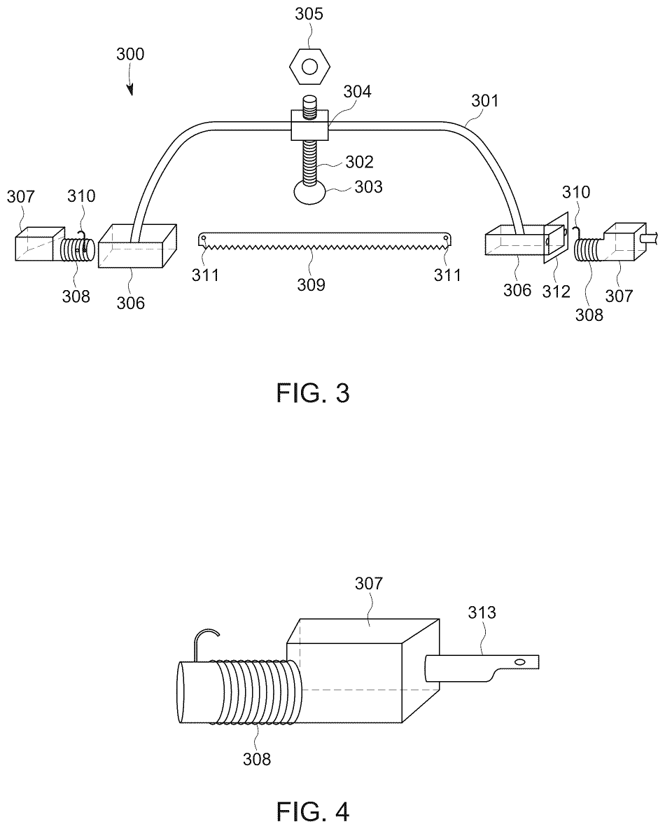

FIG. 3 shows another embodiment of a modified bow saw 300 of the disclosure. FIG. 3 shows frame 301 with adjustment screw 302 with wheel 303 and nut 304 for height adjustment. FIG. 3 shows frame 301 attached to spring holder tubes 306 at each end which spring holder tubes are configured to accommodate Rods 307. Rods 307 are configured to accommodate an attached compression spring 308. The compression springs 308 attach to blade 309. In the embodiment shown in FIG. 3, compression springs 308 are attached to the blade at each end via hooks 310. The blade 309 in FIG. 3 is shown to include holes 311 at each end to connect to the hooks. Rods 307 are configured to be attached to spring holder tubes 306.

As shown in FIG. 3, in this embodiment, the rod has an elongated portion with spring attached and a section which is geometrically configured or matched to connect to the spring holder tube. In FIG. 3, the rod is firmly attached to the tube via plate 312. For example, the connection may be made by screws. The same tube, rod with attached spring, and hook configuration may be present at both ends of the blade and frame.

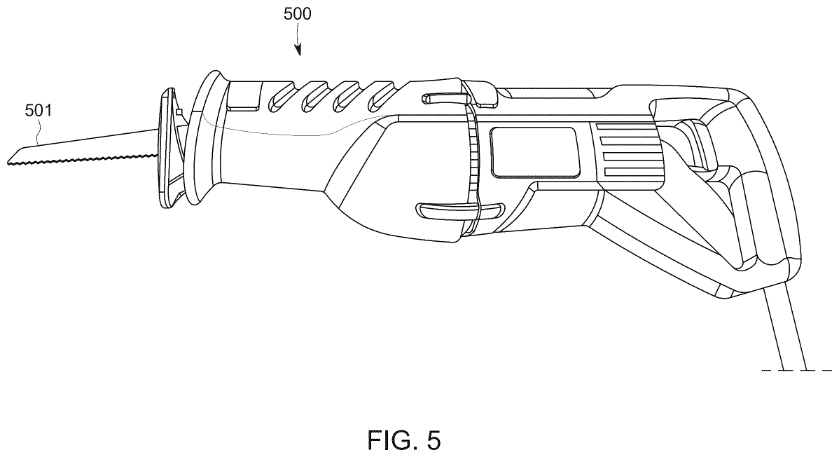

FIG. 4 shows an enlarged view of rod 307. It can be seen the rod may be rotated to provide horizontal or vertical cuts. FIG. 4 further shows a reciprocating saw attachment adapter 313. The reciprocating saw attachment will generally have a standard attachment mechanism to permit attachment to a reciprocating saw.

FIG. 5 shows an example reciprocating saw.

The disclosure further provides methods of using a reciprocating saw to drive or power a modified bow saw of the invention. The methods include attaching a modified bow saw to a reciprocating saw via a standard reciprocating saw attachment mechanism. The reciprocating saw is then powered and the push and pull action of the reciprocating saw is used to drive the modified bow saw blade back and forth (in a sawing motion) via the springs.

As such, the methods include attaching a modified bow saw of the disclosure to a reciprocating saw; powering the reciprocating saw; and sawing a material with the modified bow saw blade and attached springs.

Materials that can be sawed include wood, bone, plastic, metal, construction materials, etc., or generally speaking, any material that can be cut with a bow saw or a hacksaw.

### Glossary

A bow saw as used herein, is a hand saw including a frame with a saw blade attached to two ends. The frame can be of any convenient shape as long as two ends are connected to the saw blade. Typical shapes for the frame include a rectangular shape with one of the lengths being the saw blade. Other shapes for the frame can include a C-shape, elliptical, a semicircular shape, a semi-oval shape, or any other shape used for a bow saw. For example, other shapes could include a parallelogram or trapezoidal shape with one of the lengths being the blade.

A bow saw blade as used herein is a cutting or saw blade and is usually made of steel, carbon steel, bi-metal, or carbide and typically has a toothed blade for cutting. In embodiments, the length of a bow saw blade can range from about 12 inches to about 36 inches. A preferred embodiment may be about 21 inches long.

A spring holder as referred to herein is generally a rigid or stationary component with respect to the frame, is attached to the frame, and is configured to hold a spring. The spring holder may be, for example, rectangular in shape to accommodate an elongated spring. In other embodiments, the spring holder can be cylindrical or tubular or any other convenient shape to hold the spring as described herein. The spring holder is attached to the frame and is configured to hold a spring that moves (compresses and expands) within the stationary or rigid spring holder attached to the frame.

Springs as used herein, are attached to each end of the saw blade. The attachments can be made of any convenient method that securely holds a spring to a saw blade. For example, as shown in the drawings, connections can be made through a hook. Other attachment mechanisms can also be used including welding, screws, nuts and bolts, etc. A spring is generally made from rigid material (typically metal) that is bent or molded into a coil shape and that returns to its original shape after being compressed or extended. Springs as referred to herein would be typically manufactured from spring steel.

Compression springs are preferred. In embodiments, a compression spring may have a free (uncompressed or stretched) length of about 1 inch to about 25 inches. The inner diameter may range from about 0.3 inches to about 3 inches. Outside diameter may also range from about 0.3 to about 3 inches. Compression springs may be made from, for example, stainless steel, spring steel, beryllium copper, music wire steel, phosphor bronze, and/or be hard drawn.

An adjustable bar depth guide as used herein is generally an elongated, straight, rigid component that is attached at each end to the frame. The adjustable bar depth guide would generally be adjustable in a direction that is perpendicular to the length of the saw blade.

An adapter or adapter plate as used herein generally refers to a component configured to connect one of the springs (connected to the saw blade) to a motorized or powered circulating saw. In one embodiment, an adapter may be a flat square shaped piece with screw holes configured to allow the adapter to be connected via screws on one side to the circulating saw and on the other side to the saw blade.

Ergonomic handles are handles attached to the frame and provide for easier handling of the saw. Ergonomic handles may generally be of any appropriate shape or size configured to allow a user to handle the saw.

A reciprocating saw as used herein is a type of machine-powered saw in which the cutting action is achieved through a push-and-pull (“reciprocating”) motion. The reciprocating saw's reciprocating mechanism is thus the component of the reciprocating saw that reciprocates or oscillates back and forth. An example reciprocating saw is shown in FIG. 5. In a typical hand reciprocating saw the blade can be removed or detached and other components can be connected to the reciprocating saw in place of the blade through standard attachment mechanisms. The reciprocating saws can typically be in the 4 to 12 amp range (6V to 20V) and be corded or battery-operated.

Reciprocating saw attachment sets and tools including adapters to attach a variety of tools and accessories to a reciprocating saw are commercially available. Adapters or reciprocating saw connection adapters are attached directly to the reciprocating saw's reciprocating mechanism and permit the attachment of accessories to the reciprocating saw. Such adapters are commercially available and are typically referred to as reciprocating saw attachment adapters or connectors.

As described herein, and shown in the drawings, in this case an attachment adapter or connector can connect, for example, to the rod component holding a spring which attaches to the blade.

While this disclosure includes specific examples, it will be apparent after an understanding of the disclosure of this application has been attained that various changes in form and details may be made in these examples without departing from the spirit and scope of the claims and their equivalents.

1. A modified bow saw comprising:
a bow saw frame comprising two ends;
two spring holders, with one attached to each end of the bow saw frame;
the two spring holders each comprising a spring;
a saw blade comprising two ends;
each end of the saw blade attached to one of the springs; and
a reciprocating saw attachment adapter configured to attach one spring to a reciprocating mechanism of a reciprocating saw.

2. The modified bow saw of claim 1, wherein the frame includes an adjustable depth bar guide.

3. The modified bow saw of claim 1, wherein the frame comprises one or more ergonomic handles.

4. The modified bow saw of claim 1, further comprising one or more braces connecting the frame and the spring holder.

5. The modified bow saw of claim 1, wherein the reciprocating saw attachment adapter connects to a plate on the spring holder.

6. The modified bow saw of claim 1, wherein the saw blade comprises steel or a carbide.

7. The modified bow saw of claim 1, wherein the frame comprises steel.

8. The modified bow saw of claim 1, wherein the spring comprises steel.

9. A method of using the modified bow saw of claim 1 comprising: attaching a modified bow saw of claim 1 to a reciprocating saw via a reciprocating saw attachment adapter; powering the reciprocating saw; and sawing a material with the modified bow saw.
